(19) United States
(12) Patent Application Publication
(10) Pub. No.: US 2022/0233138 A1
(43) Pub. Date: Jul. 28, 2022

(54) INTRAORAL SCANNER APPARATUS

(71) Applicant: DENTAL SCANNER SOLUTIONS KFT., Debrecen (HU)

(72) Inventors: László Rajmund HAVASI, Szeged (HU); Attila BALOGH, Fehérgyarmat (HU)

(73) Assignee: DENTAL SCANNER SOLUTIONS KFT., Debrecen (HU)

(21) Appl. No.: 17/609,421

(22) PCT Filed: May 7, 2020
(86) PCT No.: PCT/IB2020/054318

§ 371 Date: Nov. 8, 2021

(30) Foreign Application Priority Data
May 10, 2019 (HU) U1900085

(51) Int. Cl. A61B 5/00

(52) U.S. Cl. CPC A61B 5/4547; A61B 2560/0431; A61B 5/0088

(57) ABSTRACT

A hand-held intraoral scanner apparatus includes a handle, a scanner shaft and a head unit, wherein the head unit is arranged at an end of the scanner shaft and the head unit includes at least one sensor. The hand-held intraoral scanner apparatus further includes a processing unit, a moving arrangement and at least one motor. The processing unit is electrically interconnected to the at least one motor, the moving arrangement and the at least one sensor, a first end of the moving arrangement is interconnected to the head unit, and a second end of the moving arrangement is interconnected to the at least one motor. The processing unit is configured to move the head unit via the at least one motor and the moving arrangement, wherein the head unit is configured to move around at least one of two rotation axes. The two rotation axes extend perpendicular to each other.

### CROSS REFERENCE TO THE RELATED APPLICATIONS

This application is the national stage entry of International Application No. PCT/IB2020/054318, filed on May 7, 2020, which is based upon and claims priority to Hungarian Patent Application No. U1900085, filed on May 10, 2019, the entire contents of which are incorporated herein by reference.

### TECHNICAL FIELD

The subject of the invention is an intraoral scanner apparatus that comprises a handle, a scanner shaft and a head unit arranged at the end of said scanner shaft, the head unit comprising at least one sensor.

### BACKGROUND

Digital improvements and digital imaging solutions become more and more relevant to dentistry and dental mechanics. 3D imaging and 3D printing improve quality and accuracy. Dental laboratories produce crowns, bridges, dental models, and various braces combining intraoral imaging, 3D software design and 3D printing. However, several challenges remain on the field of intraoral 3D dental scanning; the processes are slow and uncomfortable, and they require advanced manual techniques. Taken together, such challenges limit efficiency and lead to a decrease in patient satisfaction. Both parties (scanner operator and patient) are interested in using a process that is fast, comfortable, and sufficiently accurate.

The state of the art includes the following solutions. US patent document No US2017181815 describes an intraoral scanner and a procedure for its use that can be used to produce 3D surface models of dental structures, in particular to produce dental prosthetics. The scanner also includes a probe head with special design.

South Korean patent document No KR20170032644 describes a 3D scanner that can be used in dental procedures. When recording intraoral images, the device is capable of selecting automatically the most suitable resolution from high and low resolution imaging options. The 3D scanner includes a light source that emits light for measurement purposes; a camera unit that detects the reflection of the measuring light of the device; a control unit; a storage unit that stores image data; and a communications unit that sends and receives image data.

US patent document No US2017100225 describes an intraoral scanner that is suitable for scanning teeth and dentures; it also includes a supplementary unit that can be used for disinfection. The device may also include a mounting unit and a sensor unit that detects exhalation or humidity. The exhalation sensor is designed to detect humidity within the mounting unit, and it generates a signal for de-humidifying according to the detected humidity level. Thus, a UV sterilizing unit emits UV rays into the mounting unit in response to the sterilization signal. In response to the de-humidifying signal, a built-in ventilation unit can move air into the mounting unit.

Australian patent document No AU2017213787 describes a solution that produces 3D images of teeth; for this purpose, it also includes two handles that are suitable for recording images. The device is used intraorally. The head of the device is fitted with a ball joint that can be connected to the far end of a long handle; these together allow for longitudinal, horizontal and/or vertical movement in a 360 degree angle. The invention is not capable of turning around two axes, and it can be used for teeth only, but not for gingivae. Another disadvantage of that invention is that it does not have a warm air nozzle or a monitor.

South Korean patent document No KR20180030322 describes a procedure that uses CAD/CAM technology to produce a 3D scan of teeth for dental treatment purposes. The device also includes a light source.

German patent document No DE102016121687 describes an intraoral scanner that can be controlled using a manual controller. While controlling the device, the user can inspect the observed item and data concerning that item through the lens of glasses.

US patent document No US2018153646 describes a solution that uses CAD/CAM technology to produce 3D images for the primary purpose of orthodontics. The device may also include additional cameras to facilitate modelling. The use of this solution makes it also possible to map the roots of teeth. Thus, the solution handles 3D data as an input, and it produces a model of teeth, dentures, and bones relying on that input.

US patent document No US2016374784 describes a 3D intraoral scanner that uses CAD/CAM technology and is fitted with a display. The system uses cone beam computed tomography (‘CBCT’) for scanning.

International patent application No. WO2018162641A1 discloses a scanner device that is used independent from a human operator, for intra-oral scanning. The scanning device has an interchangeable mouthpiece for being positioned in the oral cavity of a patient, the mouthpiece having a hollow interior, and a scanning arm extending into the hollow interior of the mouthpiece.

Taken together, such challenges concerning intraoral 3D dental scanning limit efficiency and lead to a decrease in patient satisfaction. Both parties (scanner operator and patient) are interested in using a process that is fast, comfortable, and sufficiently accurate. The solutions representing the state of the art consist of scanners with immovable head units; when using such devices, the person operating the device needs to move around his or her patient; and a flexible wrist and manual skill is required for digitizing the entire dental arch. Another challenge posed by known solutions is that the operator needs to observe the monitor continuously when changing the position of the scanner, so that he or she can recognise scanning errors and move the scanner to any problematic area. This need for attention and occupation of eyesight makes the digitisation process even more difficult. Another disadvantage of the known solutions is that the person operating the device is solely responsible for moving the scanner. No known scanner is capable of moving its head unit and/or sensor(s). Also, compressed air is commonly used, as support for imaging, to blow away any moisture from the teeth. However, this means that the scanner needs to be put down, and that the patient is exposed to an intense and often painful sensation. Furthermore, numerous known solutions are capable of scanning teeth only, but they are unfit for examining a gingiva.

### SUMMARY

The purpose of the invention is to eliminate the shortfalls of the known solutions, and to implement an apparatus that brings together functions that make the scanning process more accurate, comfortable, and fast. The apparatus solves the following problems: it does not require manual skill from the operator, and the operator does not need to walk around his or her patient or occupy any extreme posture to hold the scanner to all teeth in an appropriate angle. Consequently, the mouth or face tissues of patients do not need to be exposed to any powerful force to fit the scanner shaft into the mouth. Thus, the objective is to implement a painless and efficient scanner apparatus that can be used appropriately for both teeth and gingivae without needing any advanced manual skill.

The inventive step is based on the recognition that a solution, which is more advantageous than the previous ones, may be created by implementing the apparatus according to claim 1. It forms also part of this recognition that the scanner apparatus needs to be fitted with a movable head. The use of a movable head makes it possible to implement a scanner apparatus that does not put any load on the wrist and does not require any advanced manual skill, while being suitable for scanning teeth, dental arches, complete dentures, and gingivae thoroughly. A movable head unit also makes it possible to move the sensor automatically.

In line with the desired purpose, the most general implementation form of the solution according to the invention may be realized according to claim 1. The various implementation forms are described in the sub-claims.

According to the objective set, the solution according to the invention is an intraoral scanner apparatus that comprises a handle, a scanner shaft and a head unit arranged at the end of said scanner shaft, and the head unit includes at least one sensor. A distinctive feature of the invention is that the apparatus is also fitted with a processing unit, a moving arrangement, and at least one motor; the processing unit is electrically interconnected to the motor, the moving arrangement and the sensor; a first end of said moving arrangement is interconnected to the head unit, and a second end of said moving arrangement is interconnected to the motor; the processing unit is configured to move the head unit via the motor and via the moving arrangement; such that the head unit is configured to move around at least one of two rotation axes, so that said rotation axes extending substantially perpendicular to each other. Movement of the head unit includes any and all kinds of movements, turns, tilts, minor angle adjustments, and angle changes and modifications.

In a further possible implementation form, the moving arrangement is arranged in the scanner shaft; the longitudinal axis of the moving arrangement is parallel to rotation axis A1, the moving arrangement includes an inner shaft and a rotating bar, the inner shaft and the longitudinal axis of the rotating bar are parallel to the longitudinal axis of the moving arrangement, and a first end of said inner shaft is interconnected to the head unit, and a second end of said inner shaft is interconnected to the motor; the inner shaft is configured to move linearly in the direction of the longitudinal axis such that said linear movement is configured to move the head unit around rotation axis A2; and a first end of said rotating bar is interconnected to the head unit, and a second end of said rotating bar is interconnected to the motor; and the rotating bar configured to rotate around the longitudinal axis such that said rotation is configured to rotate the head unit around rotation axis A1.

In a further possible implementation form, the rotating bar is hollow, and the inner shaft is arranged inside the rotating bar; and the inner shaft is connected to the head unit through a connecting part.

In another implementation form, the apparatus comprises a control unit, said control unit is interconnected to the processing unit and the motor; and the head unit is configured to move in response to user input on the control unit. It is also possible that the movement of the head unit is controlled by an algorithm. This means that the head unit may be controlled by various means; such means may be used simultaneously or separately. The apparatus is fit for use even if only one type of control is permitted; e.g. input by the operator is not available at all. Such a scenario may be necessary, for example, where a control unit is not available, or the apparatus is used by an operator without specialized training, and the movement of the head unit is fully automatic. In another possible implementation form, the sensor can move around any of two rotation axes, even without moving the head unit.

In another implementation form, the apparatus comprises two motors; one motor is connected to the head unit through the inner shaft, and the second motor is connected to the head unit through the rotating bar. In a possible implementation form, the motors are stepper motors. Using the two-motor version is most advantageous where the head unit is capable of moving around two axes, possibly at the same time. In such a scenario, one motor moves the head unit around one rotation axis, and the second motor moves the head unit around the other rotation axis.

In a further possible implementation form, the apparatus is fitted with at least one nozzle, a fan, and a pipe, and the fan is connected to at least one nozzle through the pipe, and the nozzle is located in the scanner shaft.

In a further possible implementation form, the apparatus is covered by a cover, and the fan is installed in the cover.

In a further possible implementation form, the apparatus comprises a monitor that is built into the handle and/or is a smartphone, tablet, or digital eyeglass connected to the processing unit via wired or wireless connection.

In a further possible implementation form, the apparatus comprises a battery. The battery can be built into the handle.

In a further possible implementation form, at least one sensor is connected to the processing unit with a sensor cable, and the sensor cable is arranged in the scanner shaft.

In a further possible implementation form, the apparatus comprises a heater, and the heater is connected to the processing unit and located in the handle.

In a further possible implementation form, the apparatus comprises a thermometer, and the thermometer is connected to the processing unit with a pipe.

In a further possible implementation form, the processing unit is built-in, and it is connected to an external computing unit by wireless means.

In a further possible implementation form, the motors are placed into a support structure.

### BRIEF DESCRIPTION OF THE DRAWINGS

The invention is presented in more detail using drawings of the possible implementation forms. On the attached drawings,

FIG. 1 shows a spatial drawing of an implementation form of the invention,

FIG. 2 shows a spatial drawing of the implementation form of the invention according to FIG. 1 without the cover,

FIG. 3 shows a spatial drawing of the implementation form of the invention according to FIG. 1 without the cover covering the battery,

FIG. 4 shows a spatial drawing of another implementation form of the invention,

FIG. 5 shows a spatial drawing of an internal segment of the implementation form of the invention according to FIG. 4, displaying the path taken by the air,

FIG. 6 shows a spatial drawing of an internal segment of a possible implementation form of the invention, displaying the motors,

FIG. 7 shows a spatial drawing of another internal segment of a possible implementation form of the invention, displaying various sections of the scanner shaft with the head unit and the motors at its other end,

FIG. 8 shows a spatial drawing of another internal segment of a possible implementation form of the invention, displaying the head unit and the inner shaft,

FIG. 9 shows a spatial drawing of another internal segment of a possible implementation form of the invention, displaying various sections of the scanner shaft with the head unit and the motors at its other end,

FIG. 10 shows a spatial drawing of a possible implementation form of a segment of the scanner shaft,

FIG. 11 shows the head unit of a possible implementation form of the invention, with the cover on as shown on the spatial drawing,

FIG. 12 shows a spatial drawing of the implementation form of the invention according to FIG. 1, and

FIG. 13 shows a spatial drawing of the head unit fitted with the sensor.

### DETAILED DESCRIPTION OF THE EMBODIMENTS

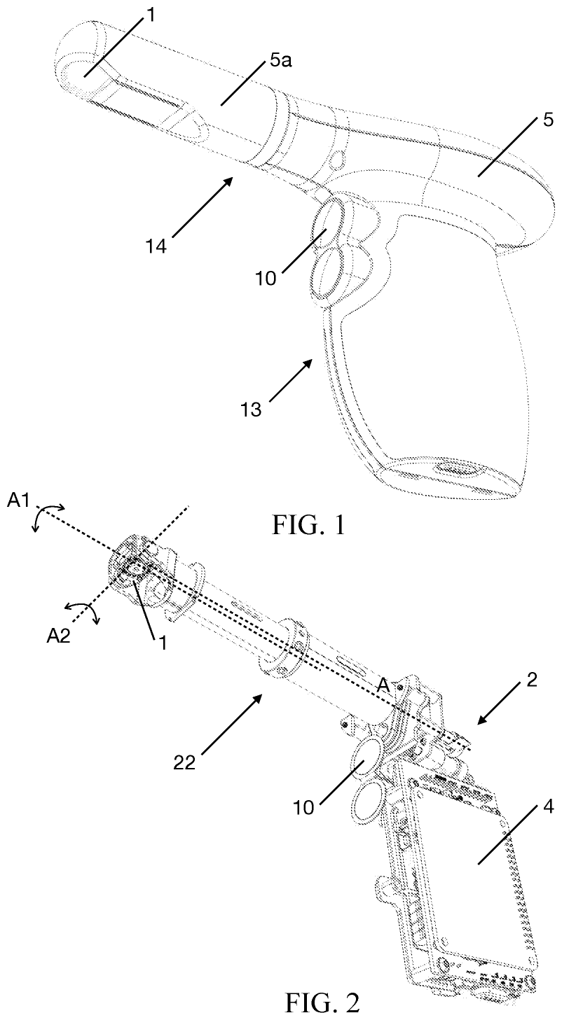

FIG. 1 shows a spatial drawing of a possible implementation form of the apparatus. In its implementation form shown on the drawing, the apparatus is portable, can be used wirelessly, and it has an approximate L shape and an ergonomic design; it includes a cover 5, a handle 13, and a scanner shaft 14. The cover 5 includes a head cover 5a that surrounds the head unit 1 and can be removed separately. Typically, the cover 5 is made of plastic. When using the apparatus to scan the mouth of a patient from the inside (e.g. for diagnostic purposes), the operator holds to handle 13 and places the scanner shaft 14, that is a part of the scanner shaft 14 as necessary, into the mouth of the patient, and he moves the head unit 1 located at the end of the scanner shaft 14 to the tooth (teeth) and/or gingiva to be scanned. As shown on FIG. 2, the apparatus also includes a processing unit 4 that is suitably built into the handle 13 below the cover 5. The processing unit 4 includes the advanced interfaces and the connecting areas for peripherals and interfaces, and the imaging sensors and the related sensor cable is also connected to this unit. The head unit 1 can move and/or turn around at least one of two rotation axes A1, A2. Suitably, the head unit 1 can move around both rotation axes A1, A2, possibly simultaneously. Movement around rotation axis A2 resembles most to a tilt, while movement around rotation axis A1 constitutes typically rotation. The head unit 1 can move and rotate around rotation axis A1 at a full 360 degree angle, and it can move and tilt around rotation axis A2 at a +−60 degree angle. When the head unit 1 turns around rotation axis A1, the head cover 5a turns together with the head unit 1. The operator can initiate and control the rotation and movement of the head unit 1 around the two axes, for example, using the control unit 10 implemented on the handle 13. However, the control unit 10 is not mandatory. Various sensors can be built into the head unit 1. The control unit 10 may include buttons, switches, mini control sticks, or pressure-sensitive areas. In its most advantageous implementation form, as also shown on FIG. 2, the control unit 10 includes two pressure-sensitive areas, as rotation in two different directions can be controlled using two separate areas. Another advantage of this implementation form is that applying pressure does not require any manual skill, and the fingers are to be kept in the same position as they are when the apparatus is held in hand. The control unit 10 is to be implemented so that its use does not require any manual skill from the operator, and the hand is not moved during use. The signals of the control unit 10 are received by the built-in processing unit, and the unit transmits the commands to the motors. Optionally, the apparatus is fitted with a built-in or external monitor, so that the operator can observe the images recorded by the head unit 1 and the information provided by the scanner during the processing steps. Such an external monitor can show the current progress status of the scanner on the denture, and if it needs to be moved to generate an outcome of adequate quality. The external monitor can be, for example, a mobile phone, tablet, or digital eyeglasses, such as VR (Virtual Reality), AR (Augmented Reality), or MR (Mixed Reality) glasses. Suitably, such items are connected to the apparatus by wireless means. A fully wireless mode can provide the highest level of comfort during use, as the operator does not need to keep moving around with or take into consideration the length of any cable. The apparatus can also operate without a control unit 10; in such a scenario, the movement of the head unit 1 is controlled by software, e.g. in a pre-programmed manner. In such a scenario, the operator only needs to place the apparatus into the mouth of the patient, and then the movements of the head unit 1 around rotation axes A1, A2 are controlled remotely and/or automatically and/or using software.

In its implementation form shown on FIG. 1, the apparatus is not fitted with a nozzle, a monitor, or a fan, as those items are merely optional. When using an apparatus without a nozzle, compressed air is applied before scanning to dry the surface of teeth, thereby improving the quality of scanning and accelerating the process. Scanning is also possible when there is moisture on the surface of the teeth, but it takes longer as the 3D calculations are more complicated. When using an apparatus without a monitor, the apparatus transmits the images through Wi-Fi to, for example, a mobile phone, and the operator can mount the mobile phone to any desired location. It may also be mounted on his or her wrist, to a tray holding the apparatus, or even to the cover 5 of the apparatus itself. This way, even a mobile phone or tablet can be used as a monitor.

The dimensions of the apparatus allow the operator to hold the apparatus in hand easily and comfortably, move it inside the mouth of a patient without any pain, and have access to any tooth. In a preferred embodiment, he apparatus is suitably implemented with the following dimensions. Handle 13 height: suitably 12 to 15 cm, but 13 to 14 cm is even more advantageous; scanner shaft 14 length: about 10 to 25 cm, suitably about 18 to 22 cm. The width of the scanner shaft 14 varies; it becomes narrower and narrower, so that it can fit into and move around painlessly in the mouth of a patient. At its widest section, the width of the scanner shaft 14 is about 5 to 10 cm, suitably 6 to 7 cm, and the width of the narrower sections may be even less than 3 cm. The diameter of the head unit 1, together with the head cover 5a (as it is inserted into the mouth of a patient), is 1 to 4 cm, advantageously 1 to 3 cm, or even more advantageously 2 to 3 cm. The length of the removable head cover 5a is about 5 to 12 cm, advantageously 7 to 9 cm, out of which the length of the section with a narrow cross-section is about 4 to 5 cm.

FIG. 2 shows a spatial drawing of the implementation form according to FIG. 1 without a cover, so that the processing unit 4 is also visible clearly. The rotation axes A1, A2 around which the head unit 1 can turn or tilt are also indicated on the drawing. The drawing also shows the moving arrangement 22, which moves the head unit 1 in various directions, and the longitudinal axis A of the moving arrangement 22. In this implementation form, the operator can control the movement of the head unit 1 using the control unit 10; minor corrections may also be carried out by software or in an automated manner. As noted above, the head unit 1 is moved by the moving arrangement 22; the functioning of multiple parts is necessary for this effect. In a most typical scenario, the control unit 10 issues an order to move, and the order is transmitted by the processing unit 4 to at least one motor 2; then, the motor 2 moves the head unit 1 through the moving arrangement 22. The moving arrangement 22 may be any kind of mechanism that is capable of transferring bi-directional movements from the motor 2 to the head unit 1, while also being capable of enabling the head unit 1 to be moved in two directions. A possible implementation form of a moving arrangement 22 is shown on a subsequent drawing. Advantageously, two motors 2a, 2b are built into the apparatus, and two motors 2a, 2b enable the head unit 1 to move around two rotation axes. However, the apparatus may also be used with a single motor 2 only; in such a scenario, the head unit 1 is typically capable of moving around a single rotation axis only, but this may depend on the implementation form.

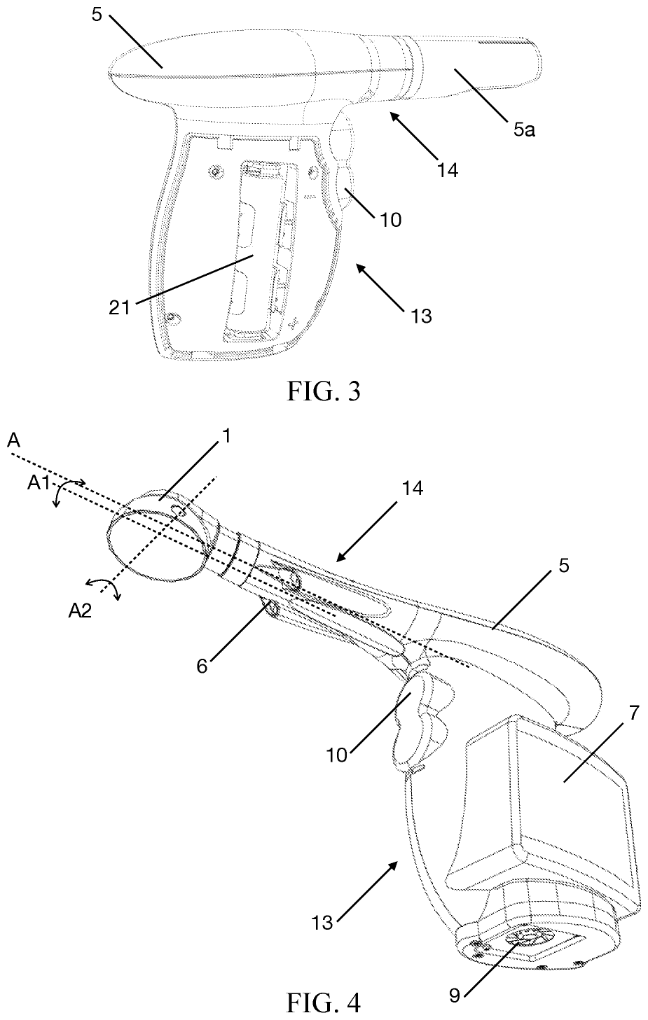

FIG. 3 shows a spatial drawing of the implementation form according to FIGS. 1 and 2. The drawing shows the apparatus with its cover 5 and head cover 5a on; only the cover section covering the battery 21 is removed, so that a possible implementation form of the battery 21 and a possible location in the handle 13 may be demonstrated. The battery 21 is, for example, a battery or an accumulator, or any unit that is able to store energy. This implementation form allows the apparatus to be fully portable and not to be connected by a wire to any power supply, dental chair, or dental working station.

FIG. 4 shows the spatial drawing of another implementation form of the apparatus. In its implementation form shown on the drawing, the apparatus has an approximately L-shaped and ergonomic design, and it includes a cover 5, a handle 13, and a scanner shaft 14. The scanner shaft 14 ends in the head unit 1, which can be tilted, turned, or tipped, and a sensor may be installed in it as shown on the subsequent drawings. Tilting and turning the head unit 1 can facilitate the production of a dental arch scan considerably. It can turn in a full 360 degree angle, and it can be tilted in a +−60 degree angle, even though tilting in a +−45 degree angle is also sufficient. In this implementation form, the scanner shaft 14 includes nozzles 6 that are capable of releasing warm air to dry the teeth; the open ends are clearly shown on the drawing. Installation of any nozzle 6 is optional; it is also possible to install only one or, as shown on the drawing, two nozzles 6. Naturally, even more nozzles 6 can also be installed. The nozzle 6 channels pre-heated air to the area to be digitized. This makes it unnecessary to use any compressed-air nozzle that might cause pain. If a nozzle 6 is installed in the apparatus, it is also advantageous to install at least one fan 9 in the apparatus. In this implementation form, air is drawn into the apparatus for the nozzles 6 by the fan 9. In this implementation form, the fan 9 is located at the bottom of the apparatus in the lower part of the handle 13, so that the hand of the operator would not obstruct the airway. The monitor 7 installed in the apparatus is located on the handle 13 of the apparatus; such an installation is advantageous as it may facilitate the use of the apparatus. The installation of a monitor 7 is optional. Installing the monitor 7 on the handle 13 can solve the problem that the operator needs to observe the denture and the monitor simultaneously, while these are otherwise located in two different directions from the operator. Looking away from the scanned area also causes hand movements, and such movements can cause problems even for experienced operators. In an implementation form without a monitor, this challenged may be solved, for example, by mounting a monitor onto the wrist of the operator. It can be difficult to resume the scanning process from the last digitized area. Thus, the monitor 7 is advantageously implemented, so that it is located in the line of sight, falling within the zone of peripheral vision. Sensor data and the status of digitization can be displayed on the monitor 7 during the scanning process. Such information is to be displayed within the field of vision, so that the information can be interpreted without moving the line of sight considerably. The monitor 7 can be foldable or installed onto the handle in a right or left hand position. The monitor 7 may be replaced or supplemented by digital glasses, such as VR (Virtual Reality), AR (augmented Reality), or MR (Mixed Reality) glasses, that display, similarly to a monitor 7, the progress of digitization on the dental arch and use colour codes to indicate areas where digitization was not carried out with sufficient accuracy. It is also possible to use a monitor 7 that is connected to the apparatus with or without a wire. The use of a built-in and movable monitor makes it possible to implement a scanner apparatus that does not put any load on the wrist and does not require any advanced manual skill. In its implementation form shown on FIG. 4, the apparatus includes a processing unit 4 located inside of the handle 13. The processing unit 4 includes the advanced interfaces and the connecting areas for peripherals and interfaces, and the imaging sensors and the related sensor cable is also connected to this unit. One of the tasks of the processing unit 4 is to send information to the monitor 7 reflecting the status of processing and scanning. For example, the processing unit 4 can provide feedback to the motors during image analysis, so that the sensor 12 is moved into the optimal position. The movement of the head unit 1, as well as the volume and temperature of the air can be controlled through the processing unit 4. Consequently, the processing unit 4 is connected to the thermometer and/or heater of the apparatus, provided that the apparatus is fitted with such supplementary units. The drawing also shows the control unit 10 on the handle 13, which can be used to control, set, or adjust the position of the head unit 1. The control unit 10 may include buttons, switches, mini control sticks, or pressure-sensitive areas. In its most advantageous implementation form, as also shown on FIG. 4, the control unit 10 includes two pressure-sensitive areas, as rotation in two different directions can be controlled using two separate areas. Another advantage of this implementation form is that applying pressure does not require any manual skill, and the fingers are to be kept in the same position as they are when the apparatus is held in hand. The control unit 10 is to be implemented so that its use does not require any manual skill from the operator, and the hand is not moved during use. The signals of the control unit 10 are received by the built-in processing unit 4, and the unit transmits the commands to the motors. If the computing capacity of the processing unit 4 is insufficient for processing all sensor data, an external computing unit may be engaged through wireless communication channels. In such a scenario, the processing unit 4 is connected to an external computing unit, suitably a smartphone, laptop, or computer, through a standard (e.g. bluetooth, 802.11b, 802.11n etc.) radio communication channel. The external computing unit may run software that processes measurement data even further. Such software may be used to carry out post-processing before a 3D model of the digital print is sent to the laboratory.

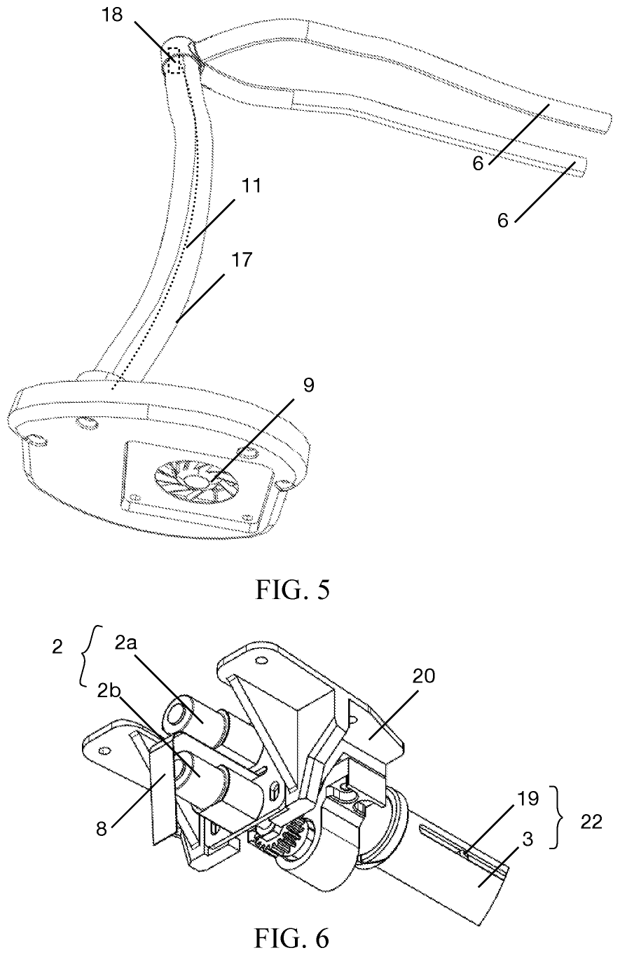

FIG. 5 shows a segment of the inside of an implementation form fitted with a fan 9 and two nozzles 6, following the path of air. The transported air is provided by a powerful built-in fan 9 sucking in air from below. The fan 9 can be installed at the bottom of the handle of the apparatus, so that it is not disturbing visually and remains uncovered even when the apparatus is held in hand. The air provided by the fan 9 enters the air pipe 17. A heater 11 and a thermostat is also located in the handle, suitably in a thicker silicone pipe, that is capable of heating the transported air to a desired temperature. The heater 11 and thermostat are optional. The heater 11 can be a Kanthal wire, for example. The top forking channels the warm air to the two nozzles 6. A photonics-based imaging process requires the scanned area to be as dry as possible. This reduces surface reflection, and the thickness of any moisture (saliva) may cause inaccurate results. Channelling the nozzles 6 to the head unit 1 allow the warm air to be focused on the scanned area directly. The warm air can be released through one or more nozzles 6. In this implementation form, two nozzles 6 are used. It is advantageous to install nozzles 6, as they can channel pre-heated air to the digitized area directly. This makes it unnecessary to use any compressed-air nozzle that might cause pain. The temperature and volume of the air released through a nozzle 6 can be controlled; for example, a thermometer 18 may be installed for this purpose, as shown in the implementation form. Suitably, the thermometer 18 can be installed at the end of the pipe 17 at the scanner shaft.

FIG. 6 shows, in a possible implementation form, segments of the apparatus that move the apparatus. In this solution, two motors 2a, 2b and other connected components, such as a rotating bar 3 connected to the motor 2b, are implemented. The drawing also shows one end of the sensor cable 8 and the support structure 20, which supports the motors 2a, 2b and may also be used to lead through the end of the sensor cable 8. The drawing also shows an implementation form of the moving arrangement 22. Typically, the moving arrangement 22 includes two parts, a rotating bar 3 and an inner shaft 19. In this implementation form, the rotating bar 3 has a circular cross-section, and it includes the inner shaft 19 that is capable of linear movement. This is advantageous because, for example, the components require less space. The motors 2a, 2b are suitably micro stepping motors; one motor 2a generates linear movement, the other motor 2b generates rotating movement. The former is responsible for tilting the head unit 1. The motor 2b transfers the rotating movement to the rotating bar 3 directly, while the other motor 2a converts rotating movement into linear movement, and then passes it on to the inner shaft 19, moving in a linear manner inside the rotating bar 3, to provide force for tilting the head unit. Thus, the motors 2a, 2b move the head unit 1 around two axes. Any space-efficient arrangement is suitable for this implementation. A possible solution is to use two micro stepping motors as shown on the drawing. The motors 2a, 2b are controlled through the processing unit 4 by the operator using the control unit 10 (in an implementation form where the apparatus is fitted with a control unit 10). Naturally, the head unit 1 may be moved around two axes using any other parts known to a professional.

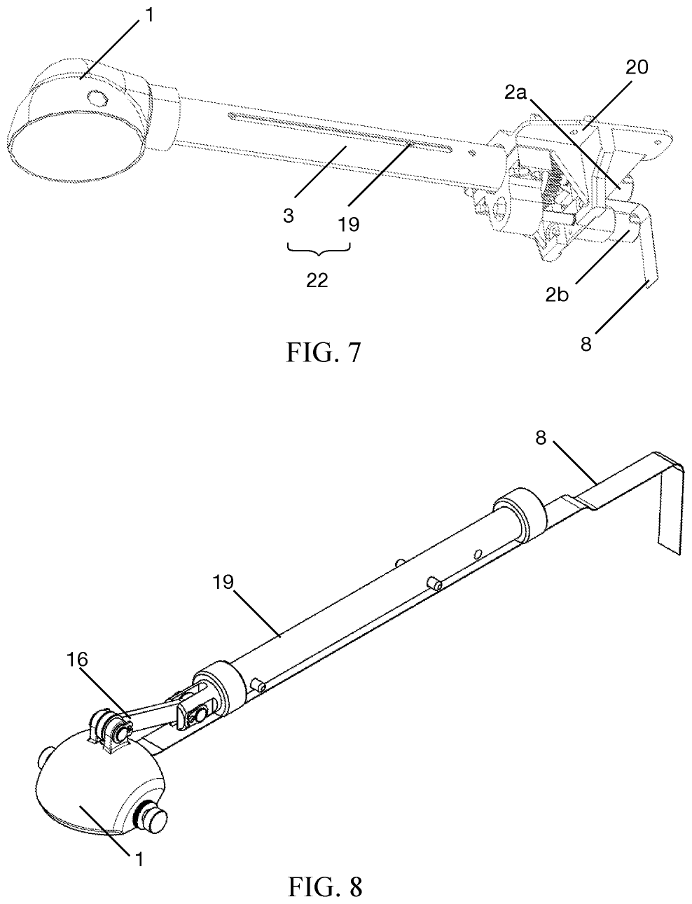

FIG. 7 shows to rotating bar 3, starting from the motors 2a, 2b located in the support structure 20 and implemented as a pipe in this example, and the head unit 1 connected to it. The sensor cable 8 and the inner shaft 19, which transfers linear movement to tilt the head unit 1, goes through the inside of the rotating bar 3. The rotating bar 3 is capable of rotating, so that it can induce a rotating movement of the head unit 1. In this implementation form, the moving arrangement 22 includes the inner shaft 19 and the rotating bar 3. These are responsible for moving the head unit 1; the motors 2a, 2b induce movement through these parts. Thus, the head unit 1 is capable of moving around two axes. The operator moves the apparatus along a path he or she considers best (or is trained to follow). This movement has a significant impact on the efficiency of scanning and the quality of the outcome. Movement can be controlled using the control unit 10, which includes two built-in buttons advantageously. Fine movement (i.e. the steps, e.g. twenty degrees) can be configured using software. The operator aims to adjust the orientation of the sensors to the scanned area. Automated controls can fine-tune such manual movement. In other words, if an algorithm detects that, for example, applying a tilt of five degrees would result in better and more accurate 3D measurement results, it can adjust the sensor 12 as necessary. However, it does not interfere to any significant extent; for example, it does not switch to scan another tooth. It merely induces movements at “micro” scale, and fine-tunes any possible clumsiness of the operator. Usability of this control mechanism is not restricted to this implementation form, and it may be used in any implementation form.

FIG. 8 also shows a spatial drawing presenting a top view of the inner shaft 19 and its connection to the head unit 1 through the connecting part 16. The inner shaft 19 tilts the head unit through the connecting part 16. The sensor cable 8 or cables run below the inner shaft 19, and they move together with the inner shaft 19. The sensor cable 8 connects the sensor 12 located in the head unit 1 to the processing unit 4.

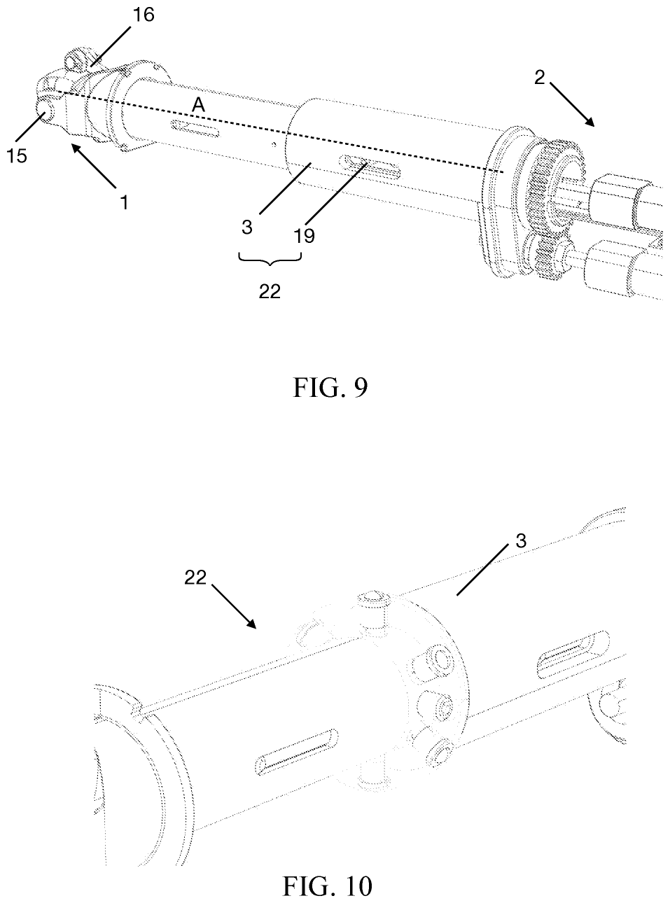

Similarly to previous figures, FIG. 9 shows an example for implementing the internal mechanism. The drawing shows the moving arrangement 22, one end of which is connected to the motors 2, and the other end of which is connected to the head unit 1. The moving arrangement 22 includes an external rotating bar 3 and an internal inner shaft 19 that is capable of linear movement. One end of the inner shaft 19 is connected to the head unit 1 through the connecting part 16, and its other end is connected to the motor 2. The inner shaft 19 is capable of moving back and forth along the longitudinal axis A. This linear movement can be restricted using the slit on the rotating bar 3; this means that the maximum length of the movement of the inner shaft 19 can be regulated by the size of the slit on the rotating bar 3. Through its linear movement, the inner shaft 19 tilts the head unit 1 back and forth using the connecting part 16, resulting in one or two degree changes, or possibly even a +−60 degree change, depending on its settings. This means that the inner shaft 19 rotates and tilts the head unit 1 along a rotation axis that is perpendicular to the longitudinal axis A. In this implementation form, both the inner shaft 19 and the rotating bar 3 include two parts; a section with a larger cross-section, and a section with a smaller cross-section.

FIG. 10 shows a spatial drawing of a section of the moving arrangement 22, where the two parts of the rotating bar 3 meet, according to FIG. 9. In a possible implementation form, two parts of the cover 5, i.e. the cover 5 and the head cover 5a, meet at this section. The head cover 5a covers the front of the moving arrangement 22 and the head unit 1. The head cover 5a can also be removed from the apparatus separately. FIG. 10 does not show the cover 5 or the head cover 5a ; it only present a possible method of mounting the head cover 5a. The drawing shows that the ball holding parts, located along the axis, make it possible to mount and dismount the head cover 5a easily and quickly. The head cover 5a can click into position easily, but its removal requires force, meaning that it does not fall off on its own. This mechanism also allows for the rotating movement of the head cover 5a. The rotating bar 3 is capable of rotating around the longitudinal axis A, and it also drives the rotation of the head unit 1. When the head unit 1 is rotating, the head cover 5a also rotates with it. Depending on the implementation of the apparatus, the maximum degree of rotation may be restricted; however, permitting full rotation is suitable, so that the head unit 1 can make a full 360 degree turn. Thus, it is possible to scan the entire denture of a patient without moving the apparatus, so that only the rotating bar 3 and the head unit 1 turn. This means that a digital image of the upper and lower teeth may be produced without causing any pain or moving the apparatus in the mouth. As seen on the previous drawing, the rotating bar 3 is connected to the head unit 1, and its other end is connected to a motor 2. Thus, the motor 2 can rotate the head unit 1 through the rotating bar 3. The inner shaft 19 and the rotating bar 3 can even move simultaneously or independently from each other, meaning that they do not depend on each other. This means that the head unit 1 can move around two rotation axes, but it is also possible that it does not move at a time, or it moves or turns along a single axis only. Naturally, this depends primarily on the scanned part of the mouth, and the anatomical structure of the patient's mouth. With a view to allowing for a most advantageous operation, the apparatus includes two motors 2a, 2b, one of which moves the head unit 1 along one axis, while the other moves the head unit 1 along the other axis.

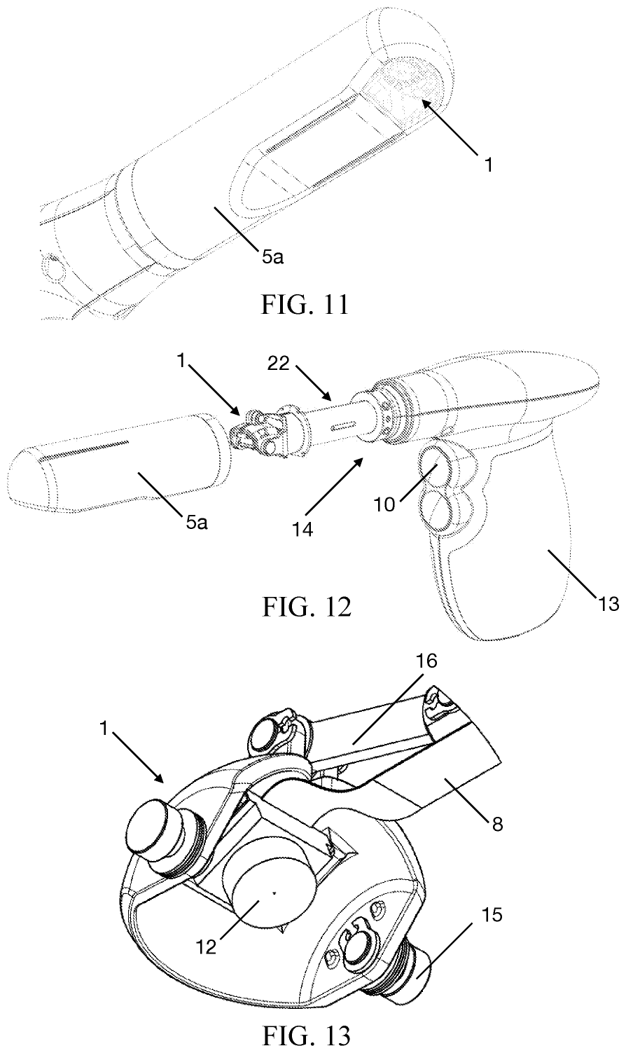

FIG. 11 shows a spatial drawing of the end of the scanner shaft 14. The drawing shows the head cover 5a covering the head unit 1. The head cover 5a is fitted with a transparent window below the sensor 12, so that it does not obstruct the scanning process. This windows also protects the head unit 1 and the patient's mouth without interfering with the scanning process. When the head unit 1 is tilted back and forth, i.e. when it turns around rotation axis A2, the head cover 5a does not move typically. However, when the head unit 1 rotates, i.e. when it turns around rotation axis A1, the head cover 5a turns together with the head unit 1.

FIG. 12 also shows a spatial drawing of the implementation form according to FIG. 1. The drawing shows as the head cover 5a, i.e. the section of the cover 5 covering the head unit 1, can also be removed from the apparatus separately. This is advantageous because it allows for cleaning and disinfection to be carried out in a faster, more efficient, and simpler manner. A possible solution for mounting the head cover 5a easily, possibly using one hand only, is shown on FIG. 10.

FIG. 13 shows the head unit 1 and the imaging sensor 12 installed in the unit. The head unit 1 is tilted by turning around the axis connecting the two mounting points located at the mounting unit 15 on the side. Such turning is caused by the linear movement of the connecting part 16, which is regulated by the inner shaft 19 performing linear movement inside the rotating bar 3. The types of sensors 12 may be installed in the head unit 1, as needed: 3D and colour imaging sensor, imaging sensor any range of sensitivity. Imaging may be carried out by 1, 3, 4, or more channel (multispectral) sensors. Also, the head unit 1 may be fitted with a lighting unit. The purpose of a lighting unit is to support facilitate and support the imaging process and the 3D measurements. The one or more sensors 12 installed are connected to the processing unit through the sensor cable 8, and, in addition to 3D measurements, they are also capable of recording correctly coloured images and the condition of the teeth (thickness of dental enamel, mineral content and condition of the dentin layer). This is important for prevention and treatment planning. The sensor 12 may also be fitted with a computing and control unit, e.g. a digital imaging sensor, and it is capable of providing information to the built-in processing unit that can be used to generate a 3D image.

The movements of the head unit 1 include both rotation and/or tilting; such movements may be carried out and controlled manually, using the control unit 10, or by software without using any control unit 10. In a most typical scenario, manual and software-controlled (automated) adjustment is applied simultaneously; suitably, software-controlled fine-tuning is performed after manual control. Typically, the head unit 1 is turned and moved around the two axes, either manually or by software, after the operator moved the sensor 12 inside the mouth to the area to be scanned.

The apparatus described above has numerous advantages. The apparatus is portable, small, and can be used without wires, meaning that it is a dental device that can be used anywhere comfortably. One advantage of the invention is that the multi-function sensors located in the head unit of the apparatus provide a complex diagnosis, and they can be used for treatment planning. Another important advantage is that the apparatus can be used painlessly and in an efficient manner, and that it can be controlled without any advanced manual skill, meaning that it is available to a wider user group. The moving head unit makes it possible to scan and digitize parts that are difficult to access without requiring any difficult movement from the operator. An advantage of the monitor, which may be built into the apparatus or worn as glasses, is that it does not require the operator to look away from the scanning process, thereby reducing the possibility of errors. Thus, the advantages include comfortable use, that the head unit may be supplemented with a lighting unit, and that the arch of the denture can be followed more closely. Another advantage is that the teeth are dried by nozzles, which also increases accuracy. Another advantage is that the temperature and volume of air can be regulated. Another advantage of the apparatus is it is designed in an ergonomic manner and eases the load on the wrist(s). The apparatus can be connected to a computer, laptop, or any other computing unit in a wireless manner, thereby accelerating the speed of calculation, memory capacity etc. Moreover, such an optional external computer can also be used to carry out post-processing operations.

Typically, the invention may be used on the field of dentistry and dental mechanics.

In addition to the above examples, the invention can be implemented in other forms within the scope of protection.

What is claimed is:

1. A hand-held intraoral scanner apparatus, comprising a handle, a scanner shaft and a head unit;
wherein the head unit is arranged at an end of the scanner shaft and the head unit comprises at least one sensor;
wherein the hand-held intraoral scanner apparatus further comprising a processing unit, a moving arrangement and at least one motor;
the processing unit is electrically interconnected to the at least one motor, the moving arrangement and the at least one sensor
a first end of the moving arrangement is interconnected to the head unit, and a second end of the moving arrangement is interconnected to the at least one motor;
the processing unit is configured to move the head unit via the at least one motor and via the moving arrangement
wherein the head unit is configured to move around at least one of two rotation axes;
the two rotation axes extend substantially perpendicular to each other;
the moving arrangement is arranged in the scanner shaft;
a longitudinal axis of the moving arrangement is parallel to a first rotation axis of the two rotation axes,
the moving arrangement comprises an inner shaft and a rotating bar;
the inner shaft and a longitudinal axis of the rotating bar are parallel to the longitudinal axis of the moving arrangement, and a first end of the inner shaft is connected to the head unit through a connecting part, and a second end of the inner shaft is interconnected to the at least one motor;
the inner shaft is configured to move linearly in a direction of the longitudinal axis, wherein a linear movement is configured to move the head unit around a second rotation axis of the two rotation axes;
a first end of the rotating bar is interconnected to the head unit and a second end of the rotating bar is interconnected to the at least one motor;
the rotating bar is configured to rotate around the longitudinal axis, wherein a rotation is configured to rotate the head unit around the first rotation axis; and
the rotating bar is hollow, and the inner shaft is arranged inside the rotating bar.

2. The hand-held intraoral scanner apparatus according to claim 1, comprising a control unit, wherein the control unit is interconnected to the processing unit and the at least one motor; and the head unit is configured to move in response to a user input on the control unit.

3. The hand-held intraoral scanner apparatus according to claim 1, wherein a number of the at least one motor is two, wherein a first motor is connected to the head unit through the inner shaft, and a second motor is connected to the head unit through the rotating bar; and the first motor and the second motor are stepper motors.

4. The hand-held intraoral scanner apparatus according to claim 1, comprising at least one nozzle, a fan, and a pipe, wherein the fan is connected to the at least one nozzle through the pipe, and the at least one nozzle is arranged in the scanner shaft.

5. The hand-held intraoral scanner apparatus according to claim 4, wherein the hand-held intraoral apparatus is covered by a cover, and the fan is installed in the cover.

6. The hand-held intraoral scanner apparatus according to claim 1, comprising a monitor,
wherein the monitor is built into the handle and/or the monitor is a smartphone or a digital eyeglass connected to the processing unit.

7. The hand-held intraoral scanner apparatus according to claim 1, comprising a battery, wherein the battery is built into the handle.

8. The hand-held intraoral scanner apparatus according to claim 1, wherein the at least one sensor is connected to the processing unit with a sensor cable, and the sensor cable is arranged in the scanner shaft.

9. The hand-held intraoral scanner apparatus according to claim 1, comprising a heater, wherein the heater is connected to the processing unit and arranged in the handle.

10. The hand-held intraoral scanner apparatus according to claim 1, comprises comprising a thermometer, wherein the thermometer is connected to the processing unit with a pipe.

11. The hand-held intraoral scanner apparatus according to claim 1, wherein the processing unit is built-in, and the processing unit is connected to an external computing unit by wireless means.

12. The hand-held intraoral scanner apparatus according to claim 31, wherein the two motors are placed into a support structure.

13. The hand-held intraoral scanner apparatus according to claim 2, wherein a number of the at least one motor is two, wherein a first motor is connected to the head unit through the inner shaft, and a second motor is connected to the head unit through the rotating bar; and the first motor and the second motor are stepper motors.

14. The hand-held intraoral scanner apparatus according to claim 2, comprising at least one nozzle, a fan, and a pipe, wherein the fan is connected to the at least one nozzle through the pipe, and the at least one nozzle is arranged in the scanner shaft.

15. The hand-held intraoral scanner apparatus according to claim 3, comprising at least one nozzle, a fan, and a pipe, wherein the fan is connected to the at least one nozzle through the pipe, and the at least one nozzle is arranged in the scanner shaft.

16. The hand-held intraoral scanner apparatus according to claim 2, comprising a monitor, wherein the monitor is built into the handle and/or the monitor is a smartphone or a digital eyeglass connected to the processing unit.

17. The hand-held intraoral scanner apparatus according to claim 3, comprising a monitor, wherein the monitor is built into the handle and/or the monitor is a smartphone or a digital eyeglass connected to the processing unit.

18. The hand-held intraoral scanner apparatus according to claim 4, comprising a monitor, wherein the monitor is built into the handle and/or the monitor is a smartphone or a digital eyeglass connected to the processing unit.

19. The hand-held intraoral scanner apparatus according to claim 5, comprising a monitor, wherein the monitor is built into the handle and/or the monitor is a smartphone or a digital eyeglass connected to the processing unit.

20. The hand-held intraoral scanner apparatus according to claim 2, comprising a battery, wherein the battery is built into the handle.
